(19) United States
(12) Patent Application Publication
(10) Pub. No.: US 2002/0062121 A1
(43) Pub. Date: May 23, 2002

(54) Perfusion apparatus and methods for pharmaceutical delivery

(76) Inventors: Karl Tryggvason, Stockholm (SE); Teija Parpala-Sparman, Oulu (FI); Olavi Lukkarinen, Oulu (FI); Pirkko Kortteinen, Turku (FI)

Correspondence Address: Richard J. Minnich, Fay, Sharpe, Fagan,, Minnich & McKee, LLP, 1100 Superior Avenue, 7th Floor, Cleveland, OH, 44114-2518, US

(21) Appl. No.: 10/004,161

(22) Filed: Nov. 15, 2001

Related U.S. Application Data
(63) Continuation-in-part of application No. 09/167,894, filed on Oct. 7, 1998.
Application No. 09/167,894 is a continuation of application No. 08/761,793, filed on Dec. 6, 1996.
Application No. 08/761,793 is a continuation of application No. 08/442,189, filed on May 16, 1995, now abandoned.

(51) Int. Cl. A61M 31/00

(52) U.S. Cl. 604/522

(57) ABSTRACT

The present invention provides methods and devices for the delivery of gene therapy pharmaceuticals to a mammalian organ in situ, in vivo, ex vivo, or in vitro. In particular, the methods and devices relate to contacting a mammalian organ viral vector with a gene therapy pharmaceutical in a re-circulating, oxygenated perfusate solution for a sufficient time to provide an effective delivery of the gene therapy pharmaceutical to the target tissue.

### CROSS REFERENCES TO RELATED APPLICATIONS

This application is a continuation-in-part of U.S. Application Ser. No. 09/167,894 filed Oct. 7, 1998, which is a continuation of U.S. Application Ser. No. 08/761,793 filed Dec. 6, 1996, now U.S. Pat. No. 5,871,464, which is a continuation of U.S. Application Ser. No. 08/442,189, filed on May 16, 1995 now abandoned.

### FIELD OF THE INVENTION

The present invention relates to methods and apparatus for delivery of pharmaceuticals to target tissues in situ, in vivo, ex vivo, or in vitro.

### BACKGROUND OF THE INVENTION

Advances in recombinant-DNA technology have made introduction of therapeutic genes into somatic cells possible (Anderson, Nature 357:455-457, 1992). In recent years, several clinical trials involving human gene therapy have been accepted by regulatory agencies. The initial human gene therapy clinical trials aimed at treating both inherited diseases (such as severe combined immunodeficiency caused by lack of adenosine deaminase in peripherial T-lymphoctes, cystic fibrosis, and familial hypercholesterolemia), as well as noninherited disease such as cancer (Wolfe, Curr. Opinion in Pediatr. 6: 213-219, 1994; Sanda et al., J. Urology 44:617-624, 1994; O'Malley et al., Arch. Otolaryngol. Head Neck Surgery 119:1191-1197, 1993; Engelhardt et al., Nature Genetics 4: 27-34, 1993; Lemarchand et al., PNAS (USA) 89: 6482-6486, 1992; Jaffe et al., Nature Genetics 1:372-378, 1992).

The development of suitable, safe, and effective gene transfer systems is a major goal of research in gene therapy. Thus far, viruses have been extensively used as vectors for gene therapy. (See for example Pilewshi et al., Am. J. Physiol. 1995; 268(4 pt 1):L657-665; Prince, Pathology 1998; 30(4):335-347). For example, retroviruses have been widely used, but they can only target actively dividing cells, and do not readily accommodate large DNA inserts. Adeno-associated viruses are also limited in the ability to accommodate large inserts, yet replication defective adenoviruses have been used successfully to transfer of a variety of genes into cells in culture and in vivo. Adenoviruses can accommodate larger inserts than retroviruses, but extra-chromosomal expression usually lasts only for a few weeks. Herpes viruses have been exploited for specific gene transfer trials into the central nervous system. Herpes viruses can carry large foreign DNA inserts, and may remain latent for long periods of time.

In spite of the availability of replication defective viruses, concerns about the safety and efficiency of such viral vectors have generated interest in the development of non-viral gene transfer systems such as liposome-DNA complexes and receptor mediated endocytosis (Felgner P. L. et al., PNAS (USA) 84: 7413-7417, 1987; Hyde Nature 362: 250-255, 1993; Nu G. Y. J. Biol. Chem. 266: 14338, 1991).

A major hurdle for effective gene therapy is the development of methods for targeting the gene transfer to appropriate target cells and tissues. Ex vivo gene transfer into explanted cultured cells and implantation of the treated cells has been used for the treatment of hematopoietic tissues (U.S. Pat. No. 5,399,346, hereby incorporated by reference), and bronchial epithelial cells in animal model. (Engelhardt et al., Nat. Genet. 1993; 4:27-34) Also, direct injection into brain and lung tumors (Cusack et al., Cancer Gene Ther. 1996; 3(4):245-249), intravenous or intra-arterial administration (Schachtner et al., Circ. Res. 1995; 76:701-708), inhalation (Katkin et al., Hum. Gene Ther. 1995; 6:985-995), and topical application (Pilewshi et al., Am. J. Physiol. 1995; 268(4 pt 1):L657-665) have been used. Major drawbacks to all of these methods are that the transduction is not highly selective, significant amounts of the therapeutic gene containing vector may be needed, and efficiency of the gene transfer is severely limited by the constraints of vector concentration, time of exposure to the target, and effectiveness of the gene transfer vector.

Much research is being conducted to enhance transgene expression in target cells. Gene transfer efficiency has been reported to improve by pretreatment with host barrier properties modificating agents (e.g polidocanol), before vector administration. (Parsons et al., Hum. Gene Ther. Dec. 10, 1998; 9(18):2661-72). Modification of the host's immune system may enhance the transgene expression in viral mediated gene transfer. (Ghia et al., Transplantation Dec. 15, 1998; 66(11):1545-51) Another method reported to enhance gene transfer efficacy is prolonging the incubation time with the vector and the target cells. (Zabner et al., J. Virol. 1996; 70;6994-7003)

One area of active research is gene therapy into mammalian kidneys, but the results have been disappointing because of poor gene transfer efficiency (Woolf et al., Kidney Int. 43: Suppl. 39: S116-S119, 1993). Moullier et al. showed some adenovirus-mediated transfer of lacZ gene into rat tubular, but not glomerular cells, following a combination of virus infusion into the renal artery and retrograde infusion into the vector (Kidney Int. 45: 1220-1225, 1994). Simple infusion of soluble virus does not appear to be an efficient transfer system. Better results were obtained by Tomita et al., (Biochem. Biophys. Res. Commun. 186: 129-134, 1992), who infused a complex of Sendai virus and liposomes into the rat renal artery in vivo, resulting in marker gene expression in about 15% of the glomerular cells.

Another active research area is gene therapy into the lung. To date, most gene therapy approaches to both inherited and acquired lung diseases have involved viral or liposome mediated gene delivery via the airway, which provides direct access to lung epithelia. (Griesenbach et al., Gene Ther. 1998; 5:181-188) At least one drawback of the aerosol delivery, especially in advanced cystic fibrosis (CF), is that the infected mucus layer in bronchioles may impair access to the cell surface. So far, intravascular infusions of the vectors have yielded quite inefficient gene transduction.

There are a variety of diseases that are candidates for somatic lung directed gene therapy, including CF and α1-antitrypsin deficiency, which are the most common inherited diseases having serious pulmonary manifestations. The first reports of in vitro correction of the CF chloride channel defect came in 1990 (Drumm et al., Cell 1990; 62:1227-1233) and in vivo CF gene expression could be established in the airways of mice in 1992. (Rosenfeld et al., Cell 1992; 68:143-155) Another candidate for lung directed gene therapy is the surfactant protein B deficiency, an autosomal recessive pulmonary disease, which manifests in neonates and leads to lethal respiratory failure within the first year of life. Gene therapy is also being considered for the treatment of inflammatory and infectious diseases and of cancer of the lung. (Dubinett et al., Hematol. Oncol. Clin. North Am 1998; 12(3):569-94)

It would be extremely beneficial to the medical arts to have apparatuses and methods for the efficient administration of gene therapy to target cells and tissues that overcome the limitations inherent to the various gene transfer vector.

### SUMMARY OF THE INVENTION

In accordance with an aspect of the present invention, there are provided methods for the administration of pharmaceuticals to targets for functional use. The term “pharmaceutical,” as used herein, includes chemical drugs, protein drugs, nucleic acid drugs, combination chemical/protein/nucelic acid drugs, and gene therapy vectors.

The term “functional use,” as used herein, includes therapeutic treatment, prophylaxis, and/or production of recombinant proteins in vivo. The term “functional use” also includes the disruption of endogenous gene expression including the use of antisense, triplex forming, catalytic and otherwise disruptive pharmaceuticals. The term “functional use” also includes the expression of recombinant proteins in target tissues, whether of endogenous or exogenous origin. The term “target,” as used herein, includes cells, tissues and/or organs.

The term “gene therapy vector” is meant to include nucleic acid constructs which are single, double or triplex stranded, linear or circular, that are expressible or non-expressible constructs which can either encode for and express a functional protein, or fragment thereof, or interfere with the normal expression of a target gene, gene transfer and/or expression vectors.

The administration of pharmaceuticals may take place where the target is in situ in a living subject. The administration may also take place wherein the target is first removed from a subject, manipulated ex vivo, and returned to the original or, alternatively, to a second recipient subject. In a preferred embodiment, the target is situated such that the circulation of the blood supply into and out of the target is relatively isolated. There are of course certain amounts of limited leakage due to small blood and lymphatic vessels.

The methods of the instant invention allow for a prolonged period of administration of pharmaceuticals to a target by way of re-circulating a pharmaceutical containing solution through the target such that a perfusion effect occurs. The methods of the instant invention allow for prolonged administration because of the unique use of the perfusion method and the oxygenation of the pharmaceutical containing solution. In one embodiment, the perfusion apparatus and target forms a closed system whereby the pharmaceuticals are administered at a starting concentration and not adjusted during the time course of treatment. In another embodiment, the pharmaceutical concentration is periodically adjusted so as to maintain or otherwise alter the concentration of pharmaceutical in the solution, or additional pharmaceuticals are added. In one embodiment, the solution does not require replenishment during the course of treatment. In another embodiment, the solution volume can be replenished as leakage or other forms of loss occur during the course of treatment. (The term “solution,” as used herein refers to the medium in which the pharmaceutical is suspended, dissolved or otherwise maintained for delivery to the target, aka. the perfusate, and includes blood, serum, plasma, saline, and/or buffered solutions.) In an additional embodiment, 350 ml of perfusate contains red blood cells (around 17% of hemocrit value), and can include about 25,000 IU heparin, about 20,000 IU penicillin and about 20,000 μg streptomycin in Krebs-Ringer solution in addition to the pharmaceutical.

The instant invention also provides methods for delivering viral vector gene therapy pharmaceuticals to a mammalian organ comprising contacting the mammalian organ with the viral vector gene therapy pharmaceutical in a re-circulating, oxygenated perfusate solution, and holding the solution at about 37° C. to provide effective delivery of the viral vector gene therapy pharmaceutical. In one embodiment, the mammalian organ is selected from kidney, liver, mammary glands, spleen, and lung.

In another embodiment, the present invention provides methods for the extended delivery of a pharmaceutical to mammalian lung tissue comprising contacting the mammalian lung tissue with the pharmaceutical in a re-circulating, oxygenated perfusate solution.

The instant invention further provides improved methods for gene therapy of lung disorders, comprising contacting the lung of a patient with a lung disorder with an amount of a gene therapy pharmaceutical effective for treatment of the disorder, wherein the improvement comprises contacting the patient's lung with the viral vector gene therapy pharmaceutical in a re-circulating, oxygenated perfusate solution.

The instant invention also provides for a perfusion apparatus functionally connected by a perfusate transfer system comprising, (a) a reservoir for perfusate, (b) means for propelling the perfusate through the apparatus, (c) means for oxygenation of the perfusate, and (d) means for connecting the apparatus to and from the target.

The reservoir for the perfusate can be any container that can be sterilized and used to collect perfusate from the target. The reservoir is connected to the means for transporting the perfusate through the system by means of tubing. While perfusion may occur at room temperature of 20° C., in one embodiment, the perfusion occurs at 37° C. Thus, in practice, the perfusate reservoir can be maintained at any desired temperature via, for example, a water bath.

In an embodiment where the means for propelling the perfusate is a peristaltic pump, the tubing is silicone or other such suitable pliable tubing. Where the means for propelling the perfusate is a peristaltic pump, no contact is made between the perfusate and any part of the pump directly. In the case where a pump with, for example an impeller blade is used, then the perfusate comes into direct contact with a part of the pump. In the usual configuration using a peristaltic pump, the tubing from the reservoir passes through the pump and connects with the means for oxygenating the perfusate.

The means for oxygenating the perfusate can be any form of artificial lung, or aeration device such that the perfusate is oxygenated without overt agitation and subsequent frothing. In one embodiment the means for oxygenating the perfusate is a membrane lung which consists of a length of semi-permeable tubing packed into a gas chamber into which is circulated oxygen rich gas, for oxygenating the perfusate as it passes through the length of tubing.

In general, the target is cannulated and connected to tubing connecting from the means for oxygenating the perfusate, and leading to the perfusate reservoir. In one configuration, the perfusate is pumped from a reservoir, through a means for oxygenating the perfusate, into the target, through the target, and back into the reservoir. The location of the pumping means in relation to the other components can be varied. The number of each component can also be varied.

Thus the instant invention provides for a method of administering a pharmaceutical to a target whereby the target is mostly isolated and continuously perfused with a perfusate containing the pharmaceutical, and said perfusate is recirculated and oxygenated. The instant invention provides for an apparatus for the administration of pharmaceuticals to a target comprising a perfusate reservoir, means for pumping the perfusate, means for oxygenating the perfusate, and means for connecting the components to one another, and with the target. In one embodiment the re-circulating perfusion apparatus comprises a perfusate reservoir receiving efflux perfusate from the target, connected with silicone tubing passing via a peristaltic pump to a membrane lung, connected by tubing and a catheter to a target.

### BRIEF DESCRIPTION OF THE DRAWINGS

FIG. 1 is a drawing of the perfusate re-circulation apparatus connected to a kidney.

FIG. 2 shows the expression of beta-galactosidase in porcine kidney following ex vivo perfusion with AdCMVlacZ virus.

FIG. 3 shows the expression of beta-galactosidase in porcine kidney following in vivo perfusion with AdCMVlacZ virus.

FIG. 3A is at 21× magnification,

FIG. 3B is at 86× and

FIG. 3C is at 214× magnification.

FIG. 4 shows the expression of beta-galactosidase in isolated human glomeruli infected with AdCMVlacZ virus.

FIG. 4A is at 21× magnification, and

FIG. 4B is at 429× magnification.

FIGS. 5A-5F shows the gene expression of beta-galactosidase in the lung tissue from a 60 minute closed-circuit perfusion examined after 7 days with X-gal staining.

FIG. 5A shows type I pneumocyte (arrow), magnification ×205.

FIG. 5B shows type II pneumocyte (arrow), magnification ×410.

FIG. 5C shows beta-galactosidase expression in the bronchial epithelial cells (arrows), magnification ×102.

FIG. 5D shows beta-galactosidase expression in the bronchial epithelial cells (arrows), magnification ×205.

FIG. 5E shows beta-galactosidase expression in the wall of a small arteriole, magnification ×410.

FIG. 5F shows the estimated quantity of beta-galactosidase expression by counting the clearly nuclear-dominant blue spots, here a total of 17 spots included, magnification ×102.

FIG. 6A shows beta-galactosdase protein in the alveolar epithelial cells, type I pneumocytes (arrows), immunohistochemical staining, AEC, magnification ×205.

FIG. 6B shows beta-galactosidase expression in a spindle-shaped alveolar endothelial cell (arrowhead) and in a cuboidal cell on the alveolar wall, type II pneumocyte (arrow), immunohistochemical staining, AEC, magnification ×410.

### DETAILED DESCRIPTION OF THE PREFERRED EMBODIMENTS

As described above, the instant invention provides for methods and means for greatly enhancing the efficiency of phamaceuticals for functional use, in particular with the use of gene therapy vectors.

The following experiments illustrate surgical ex vivo and in vivo kidney and lung perfusion using the methods and apparatus of the instant invention to effect highly efficient adenovirus-mediated gene transfer into glomerular and lung epithelial cells using pigs as the experimental animal. This method of gene therapy may have specific application to the treatment of kidney diseases such as Alport syndrome (Barker et al., Science 248:1224-1227, 1990; Tryggvason et al., Kidney Int. 43:38-44, 1993; Mochizuki et al., Nature Genetics 8:77-82, 1994), and lung diseases such as cystic fibrosis, α1-antitrypsin deficiency, surfactant protein B deficiency, and for the treatment of inflammatory and infectious diseases and of cancer of the lung. (Drumm et al., Cell 1990; 62:1227-1233; Rosenfeld et al., Cell 1992; 68:143-155; Dubinett et al., Hematol. Oncol. Clin. North Am. 1998; 12(3):569-94).

The following examples show that gene transfer into kidney cells after intra-arterial injection was highly insignificant. Even the use of high pharmacological amounts of a number of vasodilative agents does not noticeably improve the uptake of virus into the target kidney cells. In contrast, the organ perfusion system showed efficient gene transfer into glomeruli using both ex vivo and in vivo perfusion. The results of up to 80% transfer (ex vivo), or 75% transfer (in vivo), are in dramatic contrast to the results obtained with other methods of transfer into the kidney.

The following examples further show successful adenovirus-mediated gene transfer into the lung by using the apparatus and methods of the invention. The gene transfer by this method is highly organ specific but seems not to be very cell type specific. Bronchial epithelial and alveolar epithelial cells, which are essential to expression of the CF gene or surfactant protein B gene, showed transgene expression.

The perfusion system of the instant invention is applicable for gene transfer and pharmaceutical administration into a number of organs. The apparatus and methods are applicable when the target organ has a suitable blood circulation system. The flood of perfusate is most efficient if the organ has one end artery but it is not impossible to accomplish even though there are more than one. Organs that lend themselves to such methods include the kidney, liver, mammary glands, spleen and lung. It is even possible to apply the instant methods to isolated segments of blood vessels. Perfusion pressure should be monitored during perfusion to prevent any pressure damage in the organ.

Where the pharmaceutical agent is a gene therapy vector, the construct may functionally encode for endogenous or exogenous proteins, which can then be expressed in the target after treatment. Such gene transfer will allow for the expression of various proteins by the target tissues.

The most obvious benefit of the instant perfusion system and methods is the enhanced efficiency, the target specificity for gene transfer, and the possibility of using only a small amount of vector material. Furthermore, extracorporeal perfusion systems diminish the risk of administering a large amount of foreign genetic material into the general circulation of the subject, especially important for immunocompetent individuals.

The following examples are included for purposes of illustration so that the invention may be more readily understood and are in no way intended to limit the scope of the invention.

### EXAMPLE 1

Materials and Methods

Reporter gene virus construct: A replication defective recombinant adenovirus (AdCMVIacZ; Dr. James Wilson, Wistar Institute, U. Penn.) containing the cytomegalovirus promoter and E. coli beta-galactosidase gene as a reporter gene was used as the gene transfer vector (Engelhardt et al., supra.) The vector has been deleted of sequences in the E3, E1A and E1B regions, imparing the ability of this virus to replicate and transform non-permissive cells (Hurwitz et al., (PNAS (USA) 82: 163-167, 1985). Adenoviral stocks of recombinant virus were prepared and purified through double CsCl banding (Engelhardt et al., supra.). Titers of viral stocks were determined by plaque assay using 293 human embryonic kidney cells (ATCC CRL1573). The viral preparations were stored in 10 mM Tris-HCl, 10% glycerol at −70° C. until use. The viral preparations were tested for replication competence by extended cultivation on HeLa cells. Expression of the reporter gene was used to identify cells where successful transduction had occurred.

Experimental animals: Experimental animals were young 22-35 kg farm pigs which were treated according to institutional guidelines. In this experiment, operative gene transfer trials were made to 16 animals. The animals were under general anesthesia during the operation. Azaperon (Stresnil) (4 mg per kg) was first administered as intramuscular injection. For induction, medetomidine (Domitor) (80 μg per kg), ketamin (Ketalar) (4 mg per kg) and atropine (Atropin) (0.05 mg per kg) were given intramuscularly. Thiopenthal (Hypnostan) (5 mg per kg) was then given intravenously, the animal was intubated, and the anesthesia was continued under a combination of nitrous oxide-oxygen (1:1) and 1.5% enflurane (Efrane).

### EXAMPLE 2

Cultured Human Cells: Human endothelial cells, prepared from the iliac vein and artery of organ donors, were grown in M119 medium supplemented with 20% fetal calf serum (FCS), 100 IU/mL penicillin, 100 μg/ml streptomycin and 30 μg/ml endothelial cell growth factor (Sigma). Mesangial cells were isolated from human glomeruli and identified by light microscopy, based on their typical smooth muscle cell-like morphology, and by other immunohistochemical markers (Holthofer et al., Lab. Invest. 65: 548-557, 1991). The mesengial cells were grown in RPMI medium supplemented with 10% FCS, 100 μg/ml penicillin and 100 μg/ml streptomycin. In addition, a supplement of insulin (25 μg/ml), transfernin (25 μg/ml) and selenium (25 g/ml)(SIGMA) was added to the culture medium.

Isolation of human kidney glomeruli: Intact human glomeruli were prepared from renal cortex of histologically normal portions of the non-involved kidney poles of tumor nephrectomy specimens. The glomeruli were isolated by a standard three-stage sieving method (Misra, Am. J. Clin. Pathol. 58: 135-139, 1972; Tryggvason et al., Nephron 15: 6268, 1975), by passing renal cortex tissue sequentially through stainless steel sieves of sizes 250, 210 and 75 μm. The smallest pore size retained an almost pure glomerular fraction, which was transferred to six-well plastic plates. The glomeruli were cultured in RPMI1640 medium supplemented with 100 μg/ml penicillin, 100 μg/ml streptomycin, 10% FCS, 5 μg/ml insulin, 5 μg/ml transferrin and 5 ng/ml selenium.

In vitro gene transfer into cell lines and isolated glomeruli: Cultured human endothelial or mesangial cells were infected with the recombinant virus in a medium containing 2% FCS at multiplicity of infection (MOI) of 1 or 10 per cell. Following a 2 hour incubation, the medium was changed to complete culture medium, and the cells were grown for an additional 72 hours and stained with X-gal (5-bromo-4-chloro-3indolyl beta-galactopyranoside) to study the transfer and expression of the lacZ reporter gene.

The isolated glomeruli were infected with MOI 10³-10⁷ per single glomerulus by incubating the glomeruli with the virus in the supplemented culture medium in the presence of 1% FCS. Ten mM Tris was used as a control. Following 6 or 16 hours of incubation, the glomeruli were rinsed with fresh medium to remove the remaining virus and cultured in complete medium containing 20% FCS. The glomeruli were stained cytochemically three days after infection by adding 1 mg/ml X-gal into the culture medium. Blue beta-galactosidase staining was usually visible after a 2 hour incubation at 37° C.

Results

Adenoviral gene transfer into cultured cells and isolated glomeruli: Primary cultures of human endothelial and mesangial cells that were exposed for two hours to recombinant virus exhibited strong staining for the expression of the lacZ reporter gene (data not shown). Exposure of the cells to virus at an MOI of 100 for 24 hours resulted in expression of the reporter gene in the majority of the cells with no apparent changes in cell morphology. Expression of the lacZ gene appeared about 8 hours following addition of the virus.

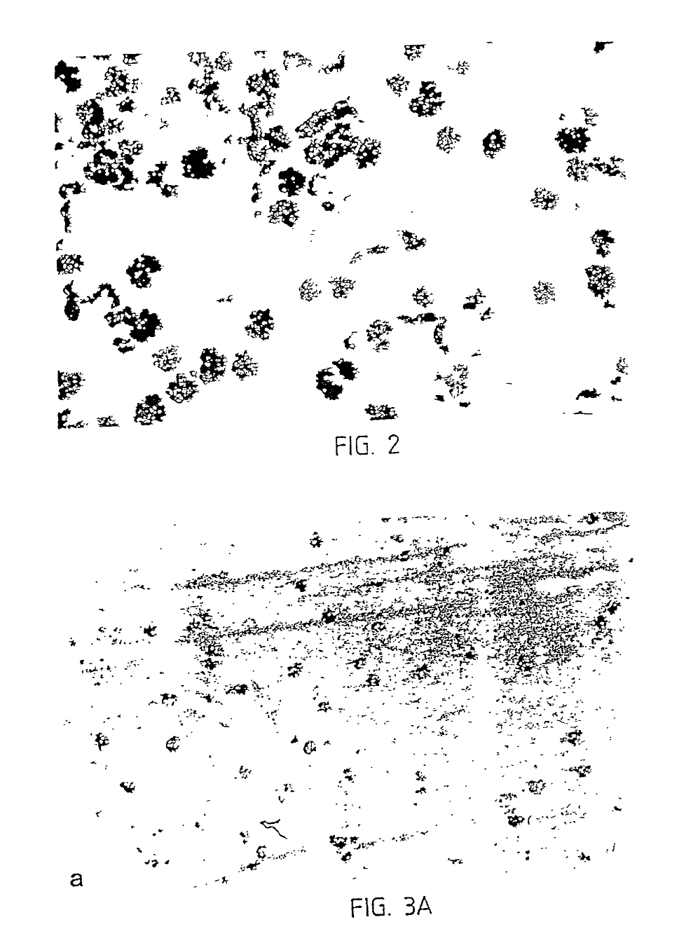

The isolated human glomeruli, some of which had lost the Bowman's capsule, could be maintained viable in culture for several days without losing their morphology. During this period, virus infection was carried out and gene transfer efficiency measured. At an MOI of about 1000 virus per glomerulus, expression of the lacZ gene was observed in practically all glomeruli, with all cell types showing positive staining reactions (FIG. 2).

FIG. 2 shows cryosections of the explanted kidney samples stained with X-gal and haematoxylin-eosin (HE) The sections show expression of the beta-galactosidase in isolated human glomeruli infected with AdCMVIacZ virus. The glomeruli were infected with the virus for about 6 hours, incubated without virus for 12 hours, and then stained with X-gal. Most of the glomeruli exhibited intense expression that appeared to involve all cell types of the glomerulus.

### EXAMPLE 3

Animals and Histological Staining: as described in Example 2.

Intra-arterial Infusion: The virus vector was injected into the renal artery during laparotomy during these tests. Eleven animals were operated on. In the first experiment, 2.5 ml (2×10⁹ pfu) of adenoviral preparation was injected through a 0.1 mm butterfly needle directly into the anterior branch of the left renal artery. The animal was sacrificed on the third postoperative day, and nephrectomy was made. Kidney samples were taken for histological examination, and expression of the lacZ gene was examined following staining with X-gal. Because the infusion was made to the lower pole of the kidney, the upper pole was used as a control. Nine of the eleven animals were operated according to a similar scheme as above, except that the vasodilative pharmacological agents were infused intra-arterially into the anterior branch of the renal artery shortly prior to infusion of the virus preparation, in an attempt to diminish the potential vascular resistance in the kidney. Five vasodilative agents were used in different trials: papaverin, alprostadil, enalapiril, verapamil and lidocain. One animal was treated as a control by infusing 10 ml of saline into the renal artery before the viral infusion. The amount of virus injected in these trials was 4×10⁹ pfu in 8 ml 0.9% saline.

Histochemical analysis: Efficiency of adenoviral gene transfer was monitored by analysis of lacZ gene expression on cryosections. Sections of 5 μm thickness were first fixed for 10 minutes in 4% gluteraldehyde in PBS. Following extensive washings with 1×PBS, the sections were incubated in a detergent solution containing 0.01% sodium deoxycholate, 0.02% NP40 and 2 mM magnesium chloride in PBS for 10 minutes. The sections were incubated in an X-gal solution (detergent solution containing 5 mM potassium ferricyanide, 5 mM potassium ferrocyanide and 1 mg/ml X-gal for 3 hours overnight at 37° C.), and subsequently counterstained with PAS. Possible cytotoxic effects of the virus were evaluated by histological examination of formaldehyde-fixed paraffin embedded tissue sections after PAS-hematoxylin staining.

Results

Gene transfer by infusion into kidney in vivo: These experiments did not lead to successful gene transfer into kidney cells. LacZ gene expression was observed in only a few scattered cells in the kidney cortex, and no signs of expression were seen in the glomeruli (data not shown). Use of vasodilative agents immediately prior to injection of the virus had no visible effect on the efficiency of gene transfer. Even use of up to three consecutive injections at 2 minute intervals did not markedly increase the efficiency of gene transfer (data not shown).

### EXAMPLE 4

Animals and Histological Staining: as described in Example 2.

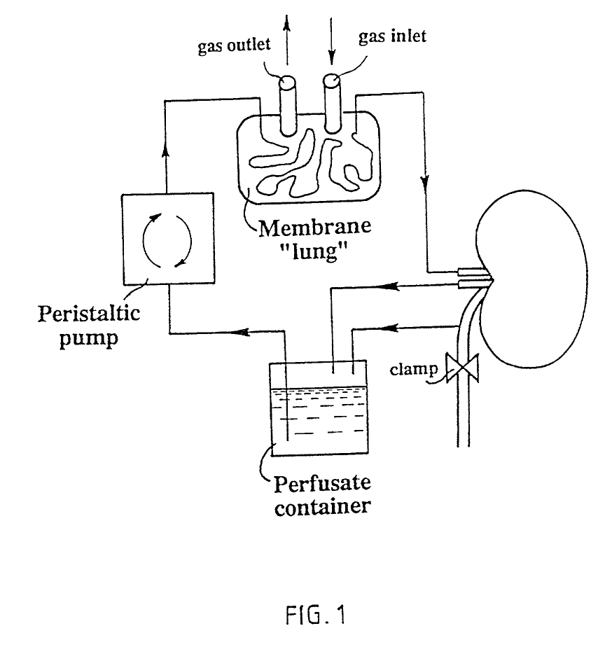

Kidney perfusion system: In order to extend the time available for infection of target kidney cells by the virus vector, an apparatus was developed which provided a closed-circuit perfusion system for the continuous circulation of virus solution in the intact kidney in vivo and ex vivo. This system, as shown in FIG. 1, consists of a reservoir for the perfusate, a pump, and an artificial membrane lung connected to the kidney to be perfused, all connected by 3 mm inside diameter silicon tubing. The reservoir for the experiment was a 300 ml glass bottle container placed in a 37° C. water bath. The peristaltic pump was from a portable organ fixation perfusion machine (PF-3A; Gambro) with a flow rate (rpm) control. The membrane lung consists of 8 m of 1.47 mm inside diameter silicon tubing in a 2000 ml glass container gassed with carbogen (95% oxygen, 5% carbon dioxide) at a pressure of 15 mm Hg, according to Hamilton et al. (J. Libid. Res. 15:182-186, 1974). The kidney was attached to the perfusion system by cannulating the renal artery with a 14 G cannula and the renal vein with a 12 G cannula. The venous and ureter effluents were collected directly into the reservoir. The perfusate had a total volume of 350 ml and contained previously separated red blood cells at a hemocrit value of 17% in Krebs-Ringer solution. Additionally, 25,000 IU heparin and antibiotics were added. For ex vivo perfusion of explanted kidneys, the perfusate also contained 20,000 IU penicillin and 20,000 μg streptomycin as antibiotics, and 5 ml of an MEM amino acid solution. For the in vivo perfusions, the perfusate contained 250 mg cefuroxim as an antibiotic. No direct measurement of perfusion pressure in the kidney was made. To circumvent this problem, the flow rate was adjusted to enable adequate diuresis. The pH and oxygen saturation in the perfusate was measured using routine laboratory “blood gas” analysis of the perfusate.

Kidney perfusion ex vivo: Before connecting the kidney to the perfusion system, a 10 ml lidocain-heparin solution (190 mg lidocain+5,000 IU heparin) and 0.9% saline were infused through the renal artery until the venous effluent was clear. The adenovirus preparation (1×10¹¹ pfu in 20 ml Krebs-Ringer solution) was then infused into the arterial inlet, and the perfusion was immediately initiated. The flow rate was set at 100-200 ml/min. Diuresis and extraction of virus into the urine were measured throughout the perfusion period.

A total of four kidneys were perfused ex vivo, the average perfusion time being 12 hours. Following the experiments, tissue samples were taken for histologic analysis as described above.

Kidney perfusion in vivo (in situ): Kidneys of three animals were perfused in vivo via laparotomy. The animals were given prophylactic antibiotic (750 mg cefuroxim) intravenously prior to the operation, and 250 mg cefuroxim was added to the perfusate. While the kidney was connected to the perfusion system, it was isolated from the systemic circulation by clamping the renal artery and vein proximally. The ureter was also clamped and ureterostomy was made to collect the effluent into the perfusate and to monitor diruesis. Since perfusion pressure in the kidney could not be measured directly, the flow rate was constantly maintained at 100-120 ml per minute. Diuresis was considered an indicator of sufficient perfusion pressure. In vivo perfusions were carried out for 60 or 120 minutes. Hydrocortisone (50 mg) was administered intramuscularly after the operation. Two animals were sacrificed on the fourth postoperative day and the appropriate kidney was removed for histological examination. One animal was maintained for up to three weeks postoperatviely, renal biopsies being taken on days 14 and 21 after which the animal was sacrificed.

Results

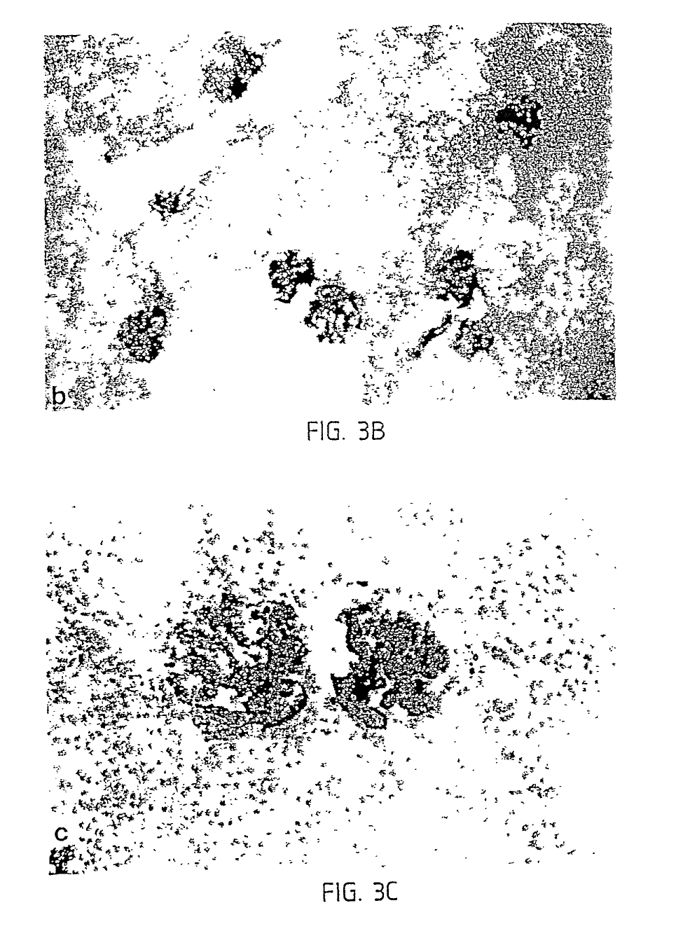

Gene transfer by kidney perfusion ex vivo: Perfusion of explanted kidneys with adenovirus at room temperature for up to 17 hours did not result in effective gene transfer to kidney cells (data not shown). In contrast, when perfusion was carried out at 37° C. for 12 hours, marked gene transfer could be seen in glomerular cells. By histologic examination, lacZ gene expression was observed in approximately 80% of the glomeruli, and in several glomeruli most mesangial and endothelial cells as well as epithileal podocytes appeared to be positive (FIG. 3). However, only little staining was seen in endothelial cells of blood vessels elsewhere in the kidney, and epithelial cells of the tubuli did not exhibit any staining.

FIG. 3 shows the expression of beta-galactosidase in porcine kidney following ex vivo perfusion with AcCMVlacZ virus. FIG. 3(A) shows the intense beta-galactosidase expression of staining in a large proportion of the glomeruli, while little if any staining was seen in other regions of the kidney (Magnification is 21×). FIG. 3(B) shows staining for beta-galactosidase in mesangial, endothelial and epithelial cells of the glomeruli, while the cells of the Bowman's capsule are negative (Magnification is 86×). FIG. 3(C) shows the expression of beta-galactosidase in two individual glomeruli. (Magnification 214×).

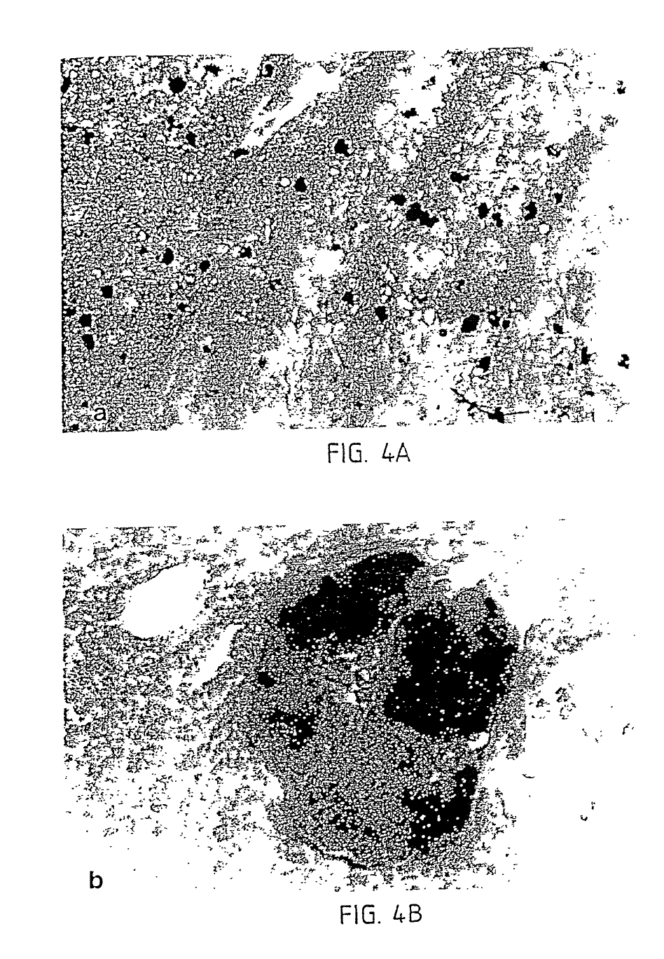

Gene transfer into kidney by perfusion in vivo: The first in vivo perfusion was carried out for 60 minutes, diuresis being normal during that period. Four days postoperatively, lacZ gene expression was seen predominately only in glomerular cells (FIG. 4). Expression was found in 9 to 45% of the glomeruli, the mean value being 27% as determined from cross-sections from experimental animals. Within the glomeruli themselves, between 2 and 50% of the cells were estimated to exhibit expression. Expression was not observed in cells of other segments of the kidney, except for some vascular endothelial cells.

Other in situ perfusions were performed for 120 minutes. There was marked polyuria, with diuresis being about 800 ml during the first hour and 1200 ml during the second hour. Four days later, lacZ gene expression was observed in 23 to 75% of the glomeruli, the mean being 58% depending upon the section. In individual glomeruli, expression was seen in most mesangial and endothelial cells, as well as in the epithelial podocytes. In certain segments, all glomeruli appeared to be positive, with seemingly all cells exhibiting expression (FIG. 4). In this experiment, expression was only seen in scattered endothelial cells elsewhere in the vascular system and epithelial cells of proximal and distal tubuli were negative.

FIG. 4 shows expression of beta-galactosidase in porcine kidney following in vivo perfusion with AdCMVIacZ virus. The kidney was perfused in situ with a closed-circuit oxygenated system at 37° C. for 2 hours. A kidney biopsy was taken 5 days later and cryosections were stained with X-gal and PAS. FIG. 4(A) shows intense expression in a large proportion of the glomeruli, while little if any staining was seen in other sections of the kidney. (Magnification ×21). FIG. 4(B) shows the staining for beta-galactosidase in mesangial, endothelial, and epithelial cells of a single glomerulus, while cells of the Bowman's capsule are negative. (Magnification ×429)

### EXAMPLE 5

Materials and Methods

Reporter gene virus: A replication defective recombinant first-generation serotype 5 adenovirus (AdCMVIacZ) containing the cytomegalovirus promoter and the E. Coli-β galactosidase gene as reporter gene was used as gene transfer vector. Sequences in the E1A, E1B and E3 regions have been deleted. Viral titers were determined by plaque assay and expressed as plaque forming units per milliliter (pfu/ml). Expression of the reporter gene serves to identify cells where successful transduction has occurred.

Experimental animals: Experimental animals were young 22-35 kg farm pigs that were treated according to institutional guidelines. Primarily, one experimental ex vivo lung perfusion was made in 37° C. for explanted lung during ten hours to give time for the possible transgene expression to become visible. In this study, the operative in vivo gene transfer trial was conducted on eleven animals. The animals were under general anesthesia during the operation. Azaperon (Stresnil: Janssen pharmaceutical) at 4 mg per kg was first administered as an intramuscular injection. For induction, medetomidine (80 μg/kg) (Domitor: Lääkefarmos), ketamin (4 mg/kg) (Ketalar, Parke-Davis), and atropine (0.05 mg/kg) (Atropin, Leiras) were given intramuscularly. Thiopenthal (Pentothal Natrium: Abbott) 5 mg per kg was then given intravenously, the animal was intubated into the left main bronchus, and the anesthesia was continued under a combination of nitrous oxide-oxygen (1:1) and 1.5% enflurane (Efrane: Abbott).

Lung perfusion system: A closed-circuit perfusion system for continuous circulation of the viral solution in the right middle lobe of the lung in vivo was used. The system consisted of a reservoir for the perfusate, a pump, an artificial membrane lung, and the lung lobe to be perfused, all connected by silicon tubing with 3 mm inside diameter as described in Example 1. The perfusions were carried out into the right middle lobe of the lungs. This lobe is triangular in shape, narrowest near the hilum, and is separated from the other lobes by this fissure.

A right thoracotomy incision was made. After dissection of the hilum, the vascular clamps were placed in the right middle lobe pulmonary artery and vein at the hilum. The lung was attached to the perfusion system by cannulating the artery of middle lobe with a 14G cannula and the vein with a 12G cannula. The venous effluent was collected directly to the reservoir. The perfusate had a total volume of 350 ml and contained previously separated red blood cells at a hematocrit value of 0.17 in Krebbs-Ringer solution. Additionally, heparin (25 000 IU) and kefuroxim (250 mg) were added as antibiotics into the perfusate. The pH and oxygen saturation in the perfusate were measured in laboratory blood gas analysis from the perfusate.

Before connecting the lung to the perfusion system, a 10 ml lidocain-heparin solution (190 mg lidocain+5 000 IU heparin in 0.9% saline) was infused through the middle lobe artery. The adenoviral preparation (1×10¹¹ pfu in 20 ml Krebbs-Ringer solution) was then infused into the arterial inlet, and the perfusion was immediately initiated. The flow rate was initially set at 100-120 ml/min, and then adjusted to meet the acquired perfusion pressure by straight pressure measurement as cm H₂O in the arterial inlet. The perfusion pressure was kept 23/20 cm H₂O, 20/15 mmHg during the perfusion procedure. Viral perfusions were carried out for 60 minutes. Thereafter, the cannules were removed, and the puncture sites in the lobar artery and vein were sutured with 7-0 nonresorbable monofilament sutures. The circulation was restored immediately. The chest wall was closed with suction chest tube in situ, which was removed during recovery from anesthesia. Hydrocortison (50 mg) was administered intramuscularly after the operation.

The animals were sacrificed on the seventh day after the perfusion, and the treated right middle lobe of the lung was examined. Lung samples prepared for histological examination, and expression of the lacZ gene was visually examined after staining with X-gal and PAS. Samples from the untreated left lung were analyzed as controls.

Histochemical analysis: Adenoviral gene transfer efficiency was monitored by visual analysis of lacz gene expression on cryosections. Ten pieces of tissue were taken randomly from the perfused lung lobe and snap frozen in liquid nitrogen. From every piece, two 5 μm thick cryostat sections were prepared for analysis. A total of 20 lung tissue sections per experiment were examined. Cryostat sections were first fixed for 10 minutes in 4% glutaraldehyde. Following extensive washings with 1×PBS, the sections were incubated in a detergent solution containing 0.01% sodium deoxycholate, 0.02% NP40 and 2 mM magnesium chloride in PBS for 10 minutes. The sections were incubated in an X-gal solution (detergent solution containing 5mM potassium ferricyanide, 5 mM potassium ferrocyanide and 1 mg/ml X-gal for 3 hours or overnight at 37° C. and subsequently counterstained with PAS (Periodic-acid shiff). Possible cytotoxic and inflammatory changes were evaluated by histological examination of formaldehyde-fixed, paraffin embedded tissue sections after PAS-hematoxylin staining.

The distribution of transgene expression was evaluated by visually examining the nuclear-dominant blue color areas of X-gal staining in the various cell types of the lung (alveolar epithelial cells, alveolar macrophages, capillary endothelial cells and airway epithelial cells). The quantification of expression was estimated by counting all uniformly stained bluish spots showing transgene expression in every 20 sections (an area of 0.50 cm² per section). The median number of spots per section and range of variation was calculated.

Results

The data demonstrated that the transgene was expressed in the alveolar epithelial cells and capillary endothelial cells (data not shown).

In the in vivo perfusion experiments, the perioperative mortality was 27%. Three animals died on the table because of arrythmia in the beginning of the experiment. Only the surviving animals were included in the study. Gene transfer into lung by closed-circuit warm perfusion in vivo was performed successfully for eight farm pigs. One perfusion was carried out for only 30 minutes and stopped earlier because of arrythmia. However this animal recovered normally from operation. The other seven perfusion experiments were carried out for 60 minutes. After recovery from anesthesia, the convalescence period of all the animals were uneventful.

The pH values in the perfusate during perfusion were between 7.40-7.43 and the oxygen saturation in the perfusate was 98.5-99%.

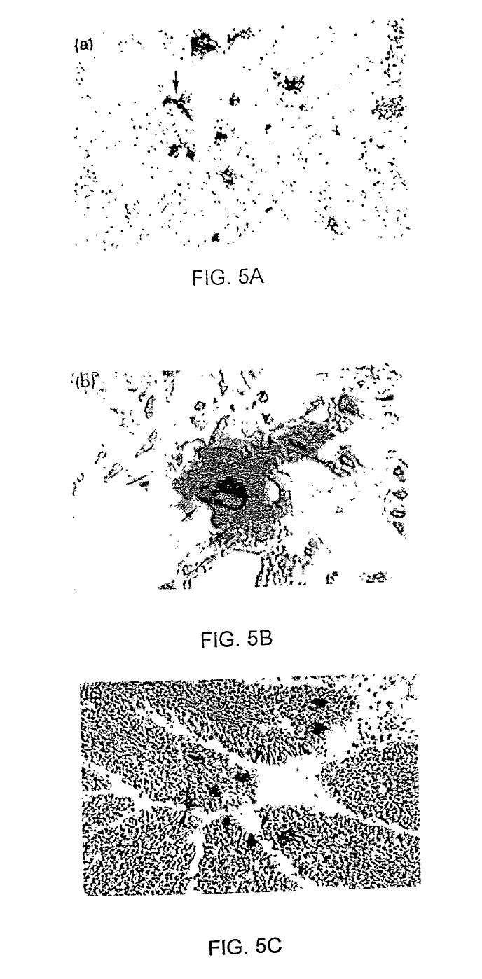

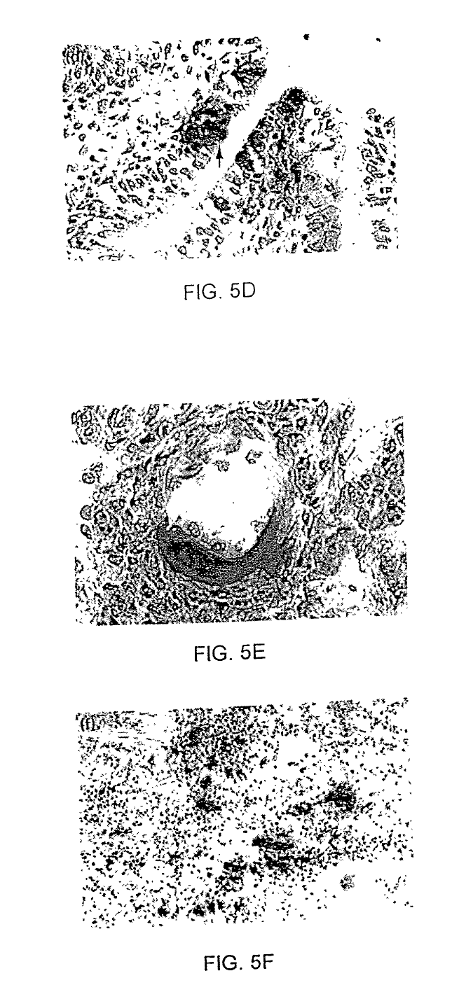

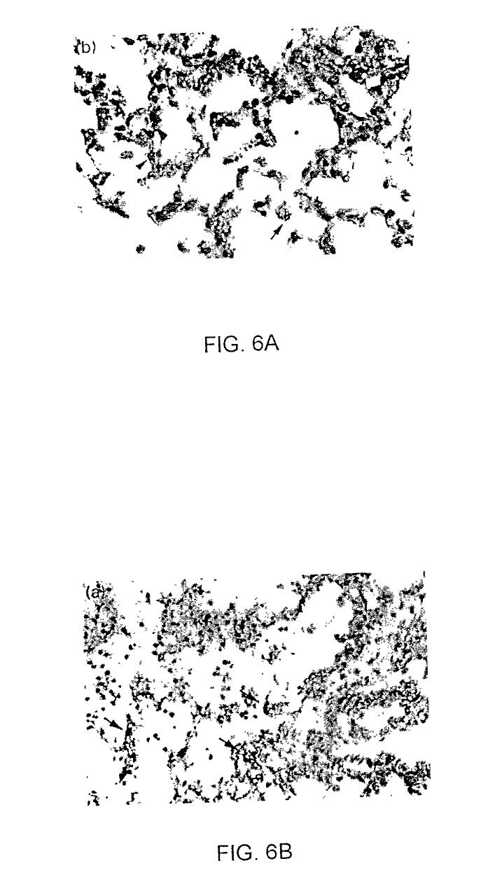

Seven days after the perfusion, the lacZ transgene expression as judged by X-gal staining was found mostly in the alveolar epithelial cells both type I and type II pneumocytes and capillary endothelial cells seven days postoperatively as shown in FIGS. 5A and 5B. The expression was also seen in bronchial epithelial cells and alveolar macrophages as shown in FIGS. 5C and 5D. Some small arterioles showed expression in their endothelial cells as shown in FIG. 5E. The immunohistochemical analysis confirmed the presence of beta-galactosidase protein in both alveolar epithelial (type I and type II pneumocytes) cells, as shown in FIG. 6A, and alveolar endothelial cells, as shown in FIG. 6B.

Transgene expression was evenly distributed throughout the specimens except on the 30 minute perfusion, which was negative. The quantification of the transgene expression was estimated by counting every nuclear-dominant blue area per section, as shown in FIG. 5F. In some places, the blue area consisted of numerous cells, but was counted as one uniformly stained single spot. The mean count of spots in the seven successful gene transfer experiments was 100 transgene expressing spots per 0.50 cm² section. The count varied between 0-365 spots per section. The detailed results of each experiment for the estimated efficiency of transgene expression by counting every blue coloured spots in specimens after X-gal and PAS staining are shown in Table 1. Twenty 0.50 cm² sections were examined per perfused lung after seven days.

TABLE 1

| | | Mean count of the transgene | |
|---|---|---|---|
| | Perfusion time/ | expressing areas/0.50 cm² | Range |
| | Minutes | section | of variation |
| I | 60 | 153 | 22-296 |
| II | 60 | 114 | 12-300 |
| III | 60 | 67 | 9-207 |
| IV | 30 | — | — |
| V | 60 | 91 | 9-217 |
| VI | 60 | 67 | 9-214 |
| VII | 60 | 185 | 104-365 |
| VIII | 60 | 22 | 3-72 |

In two experiments, the untreated left control lung showed some low intensity transgene expression in the alveolar macrophages (data not shown) but no expression was seen in the other cells of the control lung tissue.

The macroscopic appearance of the lung lobe was slightly darkish compared to other lobes seven days after the operation. Possible toxic and cytopathic effects were examined in parraffine embedded sections after haematoxylin-eosin, and periodic acid schiff stainings, and other special stainings, such Perls' iron, May-Grünwald-Giesma, van Gieson's and Verhoeff elastic stains. There was some inflammatory cell clusters, lymphocytes but remarkably less than we found in the kidney after the kidney perfusion experiments.

Discussion

The lung is an important target for gene therapy research, because this organ is often severely affected by diseases, and the pulmonary complications of the diseases are usually fatal. The lung is accessible for gene treatment by airway or intravascular routes of administration. Recombinant adenoviral vectors have been the most widely employed vehicles for gene delivery to the lung because of their natural tropism for the respiratory tract, and the overall transduction efficiency of the adenoviral vectors has been more efficient than that of cationic liposomes. Additionally, adenovirus can be grown to extremely high titers, and transfer of the recombinant gene can be accomplished in non-dividing cells.

In the present study, we have for the first time indicated the possibility of adenovirus-mediated gene transfer into the lung by continuous closed-circuit warm organ perfusion method via the vascular route. Gene transfer by this method is highly organ specific, but seems not to be very cell type specific. Significantly, bronchial epithelial cells and alveolar epithelial cells, which are the cells essential to expression of the CFTR and the surfactant protein B genes, show transgene expression. The requirements of gene transfer efficacy to correct the pulmonary manifestations of cystic fibrosis are not known exactly, but it has been estimated that even 6-10% expression in the epithelial cells may be sufficient for treatment of this disease (Alton and Geddes; Curr. Opin. Pulm. Med. 1995; 1:471-477).

The present results are particularly promising when considering potential gene therapy for different lung diseases. Cystic fibrosis is an autosomal recessive disease affecting about 1 in 3000 Caucasian births. The major cause of morbidity and mortality in CF is pulmonary disease characterized by viscous mucus secretion, chronic bacterial infection, airway inflammation and premature death at around 29 years of age. Identification of the CFTR gene has led to lung directed gene therapy research targeting into gene therapy of CF. (Alton et al., Curr. Opin. Pulm. Med. 1995; 1:471-477) Current understanding of the biology of cystic fibrosis lung disease suggests that vectors should express the transgene in mature, ciliated airway epithelia. Zabner et al. (1996) found that the ability of adenovirus vectors to express a reporter gene, and to correct defective cyclic AMP-stimulated Cl- transport in CF epithelia, was correlated inversely with the state of differentiation. The efficiency of adenovirus-mediated gene transfer could be partially corrected when the contact time between vector and epithelium was prolonged. Their observations suggest that interventions that either increase the contact time or alter the epithelium or the vector may be required to facilitate gene transfer to ciliated respiratory epithelia.

Gene therapy is also being considered for the treatment of α1-antitrypsin deficiency, an autosomal recessive disease. (Schwaiblmair et al., Drugs Aging 1998; 12(6);429-440) This disease is the second most common lethal hereditary disorder in Caucasians, with an incidence of about 1 in 6000 births. The α1-antitrypsin protein is a serum glycoprotein that is synthesized and secreted primarily from the liver. Its major site of action is the lower respiratory tract. Patients usually develop the chronic condition defined as panacinar emphysema in their fifth decade. The treatment of α1-antitrypsin protein deficiency has focused on intravenous weekly to monthly infusions of purified human α1-antitrypsin protein to maintain adequate serum levels. The limitations of this treatment include the high cost and inconvenience of long-term treatment. Gene transfer directly into the lungs or liver could correct the lung manifestations.

Human surfactant protein B deficiency represents another logical candidate for gene therapy. Surfactant protein B is a phospholipid-associated polypeptide expressed by respiratory epithelial cells. It is essential for lung function, enhancing the spreading and stability of surfactant phospholipids that reduce surface tension at the alveolar air-liquid interface. Surfactant protein B deficiency is an autosomal recessive disease of full-term newborn infants which leads to lethal respiratory failure within the first year of life.

In addition to treatment of inherited disorders, the delivery of genes via the pulmonary circulation would be a direct route for the treatment of diseases affecting vascular function, including pulmonary hypertension, pulmonary thrombosis disorders, vasculitis and of primary or metastatic lung tumors.

Gene therapy targeted to a graft offers a promising approach to the prevention of the complications after lung transplantation. (Jeppsson et al., J Throac. Cardiovasc. Surg. 1998; 115:638-643) Brochiolitis obliterans, reperfusion injury, and graft rejection are the most common complications that could be prevented by gene transfer of antiproliferative agents. Boehler et al. (Hum. Gene Ther. 1998; 9:541-551) have shown that adenovirus mediated interleukin-10 (an anti-inflammatory and immunosuppressive cytokine) gene transfer, can prevent the development of airway fibro-obliteration. Chapelier et al. (Hum. Gene Ther. 1996; 7:1837-1845) reported that ex vivo adenovirus-mediated gene transfer to the lung graft via pulmonary artery by incubation for 2 hours in 10° C. is feasible, but the efficiency was weak. Their study suggested that the gene transfer efficiency-limiting factor may be the low temperature that the graft needs for preservation during the ischemia period prior implantation. Another factor may be the lower susceptibility of the porcine model to human Ad vector. The effect of the low temperature for gene transfer efficiency is avoided by closed-circuit warm organ perfusion system used in our study. By using oxygenation of the perfusate during the perfusion, the influence of ischemia is prevented.

In summary, the examples of the present invention indicate that surgical warm organ perfusion technique is feasible for adenovirus-mediated lung gene transfer in animal models, especially into the alveolar epithelial and bronchial epithelial cells.

While the invention has been described with respect to certain specific embodiments, it will be appreciated that many modifications and changes may be made by those skilled in the art without departing from the spirit of the invention. It is intended, therefore, by the appended claims to cover all such modifications and changes as fall within the true spirit and scope of the invention.

We claim:

1. A method for the effective delivery of a viral vector gene therapy pharmaceutical to a mammalian organ comprising contacting the mammalian organ tissue with the viral vector gene therapy pharmaceutical in a re-circulating, oxygenated perfusate solution, and holding said solution at about 37° C. to provide effective delivery of the viral vector gene therapy pharmaceutical to the organ.

2. The method of claim 1, wherein the organ is in vivo and in situ.

3. The method of claim 1, wherein the organ is ex vivo.

4. The method of claim 1, wherein the organ is in vitro.

5. The method of claim 1, wherein the mammalian organ is a kidney, liver, mammary glands, spleen, or lung.

6. The method of claim 1, further comprising providing a viral vector gene therapy pharmaceutical having a promoter and an expression gene.

7. A method for the extended delivery of a gene therapy pharmaceutical to mammalian lung tissue comprising contacting the mammalian lung tissue with the gene therapy pharmaceutical in a re-circulating, oxygenated perfusate solution, and holding the perfusate solution at about 37° C. to provide effective delivery of the gene therapy pharmaceutical to the lung tissue.

8. The method of claim 7, wherein the mammalian lung tissue is in vivo and in situ.

9. The method of claim 7, wherein the mammalian lung tissue is ex vivo.

10. The method of claim 7, wherein the mammalian lung tissue is in vitro.

11. The method of claim 7, further comprising providing a viral vector gene therapy pharmaceutical having a promoter and an expression gene.

12. A method for gene therapy of lung disorders comprising contacting a lung of a patient with a lung disorder with an effective amount of a gene therapy pharmaceutical in a re-circulating, oxygenated perfusate solution, holding the perfusate solution at about 37° C., and delivering the gene therapy pharmaceutical for an amount of time that provides effective delivery of the gene therapy pharmaceutical.

13. The method of claim 12, wherein the lung disorder is selected from the group consisting of cystic fibrosis, α1-antitrypsin deficiency, surfactant protein B deficiency, pulmonary hypertension, pulmonary thrombosis disorders, vasculitis, primary lung tumors, metastatic lung tumors, brochiolitis obliterans, reperfusion injury, lung graft rejection, and combinations thereof.

14. The method of claim 12, further comprising providing a viral vector gene therapy pharmaceutical having a promoter and an expression gene.

15. The method of claim 12, wherein the target is in vivo and in situ.

16. The method of claim 12, wherein the target is ex vivo.
